(19) United States
(12) Patent Application Publication
(10) Pub. No.: US 2025/0292598 A1
(43) Pub. Date: Sep. 18, 2025

(54) A SEMI-AUTOMATIC SEGMENTATION SYSTEM FOR PARTICLE MEASUREMENTS FROM MICROSCOPY IMAGES

(71) Applicant: Merck Patent GmbH, Darmstadt (DE)

(72) Inventor: Tobias PLOETZ, Darmstadt (DE)

(73) Assignee: Merck Patent GmbH, Darmstadt (DE)

(21) Appl. No.: 18/860,780

(22) PCT Filed: Apr. 26, 2023
(86) PCT No.: PCT/EP2023/060869

§ 371 Date: Oct. 28, 2024

(30) Foreign Application Priority Data
Apr. 29, 2022 (EP) 22170766.4

(51) Int. Cl. G06V 20/69; G01N 23/2251; G01N 33/94; G06F 18/40

(52) U.S. Cl. CPC G06V 20/695; G01N 23/2251; G01N 33/94; G06F 18/41; G01N 2223/401; G01N 2223/408; G01N 2223/418; G01N 2223/421

(57) ABSTRACT

Disclosed is a method of computer-based small particle measurement for drug formulation in which digital microscopy images of the small particles used for drug formulation are created by an image sensor and provided to a computer performing a measurement software. The software segments the small particles in the digital microscopy images and calculates properties thereof, according to specific parameter sets. The software samples different candidate parameter sets, applies them automatically for the segmentation and/or calculation process, and shows the results via a display to a user. The user picks the a candidate parameter set with the best results, and the software establishes and trains an internal machine learning model with this user feedback. The software then applies the trained model to reiterate the automatic segmentation and/or calculation and user feedback obtaining process until an optimal parameter set is approved by the user.

The hereby described invention discloses a method and a system for a software based semi-automatic particle measurement.

### TECHNICAL FIELD

The invention deals with the technological area of digital image processing and drug formulation.

### BACKGROUND AND DESCRIPTION OF THE PRIOR ART

In drug formulation, especially for solid dose products, the final product, e.g. the final pill, should have certain desired properties, e.g. hardness, surface smoothness, etc. In order to tailor the production process such that these requirements are met the characteristics of the individual components need to be taken into account. For example, the surface smoothness of small API particles will influence how they agglomerate with other excipients. And the surface properties of the agglomerates will influence the properties of a pressed pill. Hence, there is a need to measure various properties of small particles and depending on the property a suitable measurement device should be chosen. Scanning electron or bright field microscopy can be used to measure the structure and fractality of the particle surface. Since microscopy images usually show many particles it is necessary to segment and isolate each particle before calculating measurements on the particle level.

There are established particle segmentation workflows implemented in open software packages like ImageJ/Fiji or Ilastik as well as commercial software such as Zeiss ZEN lite. However, these workflows require extensive training and expertise of the user. Furthermore, segmenting particles with these workflows require tuning of various parameters of the user and other user interventions for each image that should be analyzed, rendering the whole process time consuming.

For certain properties that can be assessed through microscopy images, there are also other commercial tools that rely on other measurement approaches. For example, particle size distributions can also be obtained from devices from Beckman Coulter Life Sciences or Thermofisher that employ laser diffraction. However, these do not allow for a direct visual analysis of surface properties like the surface smoothness.

### SUMMARY OF THE INVENTION

The task of this patent application is therefore to provide a more efficient particle measurement approach for drug formulation which also requires less specific user expertise about the measurement process.

This task has been solved by a method of computer-based small particle measurement for drug formulation, in which digital microscopy images of the small particles used for the drug formulation are created by an image sensor and provided to a computer performing a measurement software, the software is segmentating the small particles in the digital microscopy images and calculating properties of the small particles according to specific parameter sets, wherein the software samples different candidate parameter sets, applies them automatically for the segmentation and/or calculation process and shows the results via a display to a user, wherein the user picks the candidate parameter set with the best results, the software establishes and trains an internal machine learning model with this user feedback and applies the trained model to reiterate the automatic segmentation and/or calculation and user feedback obtaining process until an optimal parameter set has been approved by the user. Proposed is a general framework for optimizing parameters of a workflow. Specifically, the system samples different candidate parameter sets and runs the workflow automatically. The user sees the respective results, e.g. the particle segmentation corresponding to a parameter set, and picks the best one. Then the system learns from this feedback and builds an internal model about what parameter combinations are leading to good results. Then, new, more promising candidate set of parameters is sampled. Again, the user picks the best result, the system learns from this feedback, and this process is continued for a few iterations until the system has converged on a good parameter set. From a user perspective this process is quite intuitive, since the user only has to compare final results instead of tuning individual workflow parameters.

Advantageous and therefore preferred further developments of this invention emerge from the associated subclaims and from the description and the associated drawings.

One of those preferred further developments of the disclosed method comprise that Active Pharmaceutical Ingredient (API) particles are used as small particles. The invention is not restricted to API-particles though. Other suitable types of small particles can also be measured with it.

Another one of those preferred further developments of the disclosed method comprise that the measured properties comprise of the structure and fractality of the small API particle surface to determine the surface smoothness of small API particles and of the particle size distributions. Those properties which are taken from the digital images are two of several possible properties which can be used. Other ones are suitable as well, but the structure and fractality of the small API particle surface and the particle size distributions are most preferred options to be applied in the segmentation process.

Another one of those preferred further developments of the disclosed method comprise that the small particles comprise of highly variable particle shapes and sizes. That's why it is so difficult to perform a reliable segmentation process and therefore measurement. It is one of the advantages of the invented method that it can handle those different particle shapes and sizes.

Another one of those preferred further developments of the disclosed method comprise that the digital microscopy images are created under variable lighting conditions. If the measurement software cannot operate with a specific lighting setting, a different setting can be used. Or it is possible to create the digital images always with a specific set of different lighting conditions and use those different exposed digital images then for the software. The different lighting conditions can be provided by a suitable lamp installation which has to be able to illuminate the test sample with the small particles which are to be measured. Different light devices with different light sources are possible—from standard LED installations up until specific laboratory devices, emitting standard white light but also light with specific wavelengths.

Another solution to the assigned task comprises of a system to perform a computer-based small particle measurement for drug formulation comprising of a computer performing a measurement software, a display to show information to a user, means for the user to input data and/or instructions and an image sensor wherein the system is arranged to create digital microscopy images of the small particles used for the drug formulation via the image sensor, to segmentate the small particles in the digital microscopy images and calculating properties of the small particles via the software according to specific parameter sets, and wherein the software samples different candidate parameter sets, applies them automatically for the segmentation and/or calculation process and shows the results via a display to a user, wherein the user picks the candidate parameter set with the best results, the software establishes and trains an internal machine learning model with this user feedback and applies the trained model to reiterate the automatic segmentation and/or calculation and user feedback obtaining process until an optimal parameter set has been approved by the user. For this system the computer is usually a separate device, but it is also possible to use a light sensor with an integrated calculation device which is then able to perform the measurement software. In any case the different devices have to be linked via a data connection, e.g. a suitable data network like LAN, wifi, Bluetooth are other. The digital images can be stored in a memory which belongs to the computer or in a separate database which is connected via the data network.

One of the preferred further developments of the disclosed system comprise that the image sensor is an scanning electron or bright field microscope creating microscopy images as digital images. Those digital images are then stored in the memory and provided to the measurement software which analyzes them according to the invented method.

Another one of the preferred further developments of the disclosed system comprise that the software comprise of either two connected software components, one component responsible for the particle segmentation and the other one for the property calculation, or of one software component performing both tasks. Which embodiment is preferred in the end depends on the used environment, like the applied programming language, available and used software libraries and/or frameworks, the used hardware etc.

The proposed solution allows to generate particle measurements in an intuitive way with minimal user interaction and less training time. DETAILED DESCRIPTION OF THE INVENTION

The method and system according to the invention and functionally advantageous developments of those are described in more detail below with reference to the associated drawings using at least one preferred exemplary embodiment. In the drawings, elements that correspond to one another are provided with the same reference numerals.

The drawings show:

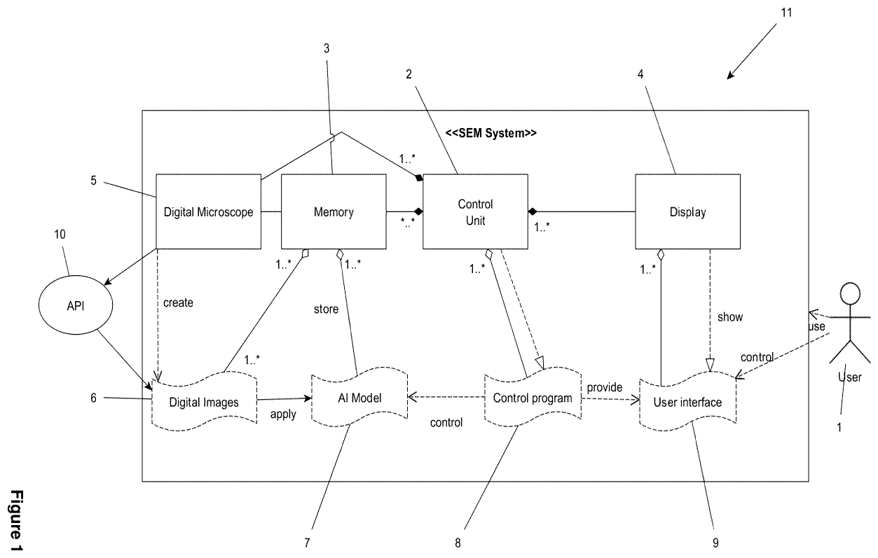

FIG. 1: A schematical overview about the used system components

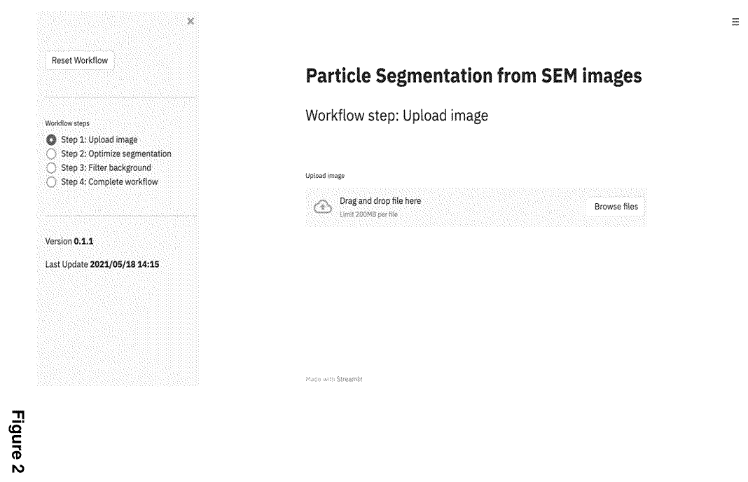

FIG. 2: Step 1: Uploading a microscopy image through a user interface

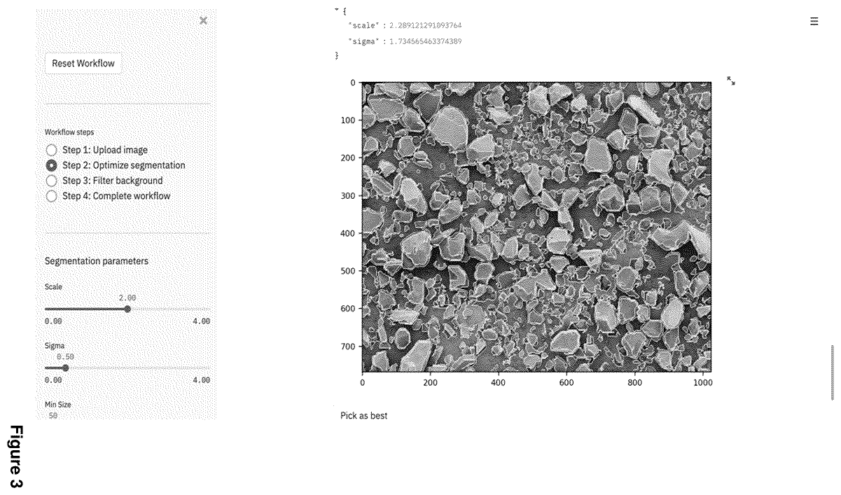

FIG. 3: Step 2a: Derive a suitable segmentation of the microscopy image

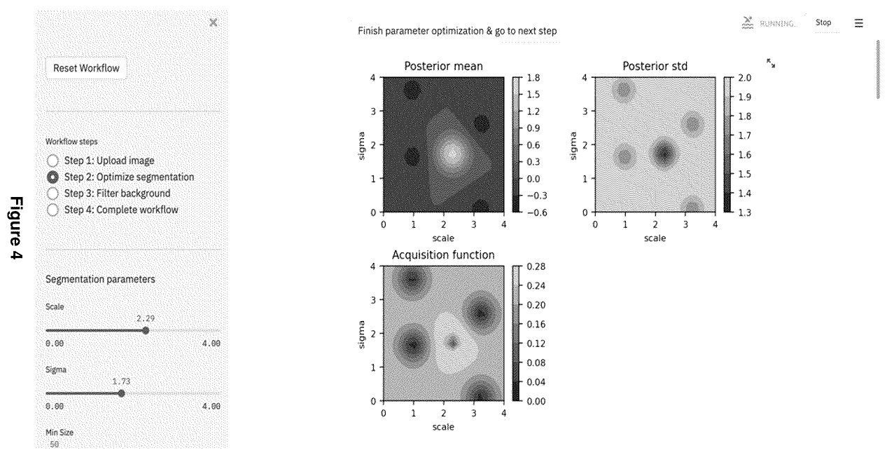

FIG. 4: Step 2b: Visualizing the progress of the internal optimization

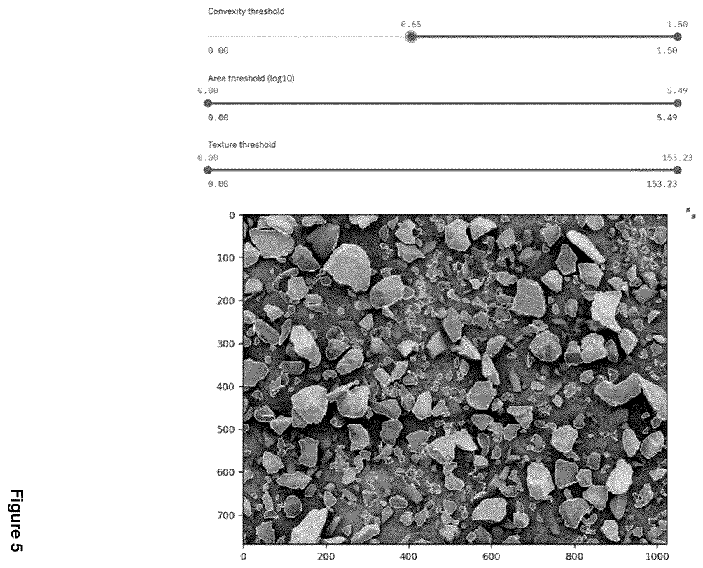

FIG. 5: Step 3: Filter background segments

The system for the Scanning Electron Microscopy (SEM) 11 in its preferred embodiment comprises of several components which are shown in FIG. 1. These components include a control unit 2 in form of any kind of suitable computer 2 which has access to a memory 3. Connected to the computer 2 is a microscope 5, preferably a scanning electron or bright field microscope, capable of creating digital microscopy images 6 of small particles 10, in particular Active Pharmaceutical Ingredient (API) particles 10 used for drug formulation of pressed pills etc. The digital microscopy images 6 are preferably stored on the memory after creation. They can also be stored on any other available memory, be it a local memory or any server like in a cloud. Also stored on preferably but not exclusively the local memory is a software 8 in form of a control program 8 which also provides a machine learning model (AI model) 7 which can be trained with and process the digital microscopy images 6. Furthermore the system 11 comprise a display which shows a User Interface 9, preferably a GUI 9, to the user 1 to whom any process relevant information, like the digital microscopy images 6 or any calculation result from the software 8 respective the AI model 7 can be shown via a display 4. The user 1 is also able to enter commands or any other data to the software 8 via the User Interface 9.

The invented method in one preferred embodiment is now shown in the FIGS. 2 to 5 and described more detailed in the following chapters.

The software application 8 performed by the described computer 2 implements the invented method. The basic workflow of this application consists therefore of three main steps:
1. Uploading a microscopy image 6 which has been created by examining a test sample with API particles 10 with a digital microscopy 5 and stored on a memory 3 through a User Interface 9. Here basic information of the uploaded image 6 gets automatically extracted (see FIG. 2)
2. Derive a suitable segmentation of the microscopy image 6 through the iterative “best candidate” selection as outlined in the previous section. To this end, the user 1 gets presented with candidate segmentations and selects the candidate that he deems best (see FIG. 3).

The progress of the internal optimization is visualized via the display 4 as shown in FIG. 4, e.g. the user 1 has feedback which parts of the parameter space are recognized to produce good segmentations. Once the user 1 is satisfied with the segmentation he proceeds to the next step.
3. The User Interface 9 allows to filter background segments based on the convexity, area and texture variance of the segments, as can be seen in FIG. 5.

Once the user 1 is satisfied with the final result, different particle measurements, e.g. size, diameter, elongation, etc, are calculated and the result can be exported—e.g. via an Excel sheet for further analysis.

In more detail, step 2 of the above mentioned workflow is based on a Bayesian optimization in the parameter space of a segmentation algorithm. Preferably, the Felzenszwalb segmentation algorithm is used but any other segmentation algorithm with a moderate amount of parameters can be used. In the Bayesian optimization framework a Gaussian process over the parameter space is used to model some form of utility of parameter sets. In this example case the Gaussian process models the quality of the resulting segmentation. Since it is desired to use pairwise comparisons as user feedback to learn the utility, the preference learning Gaussian process proposed by Chu & Ghahramani in their paper “Preference Learning with Gaussian Processes” from 2005 is leveraged. The goal of the optimization is then to find a set of parameters of the segmentation algorithm that leads to a good segmentation of physical particles in the digital input image 6. To this end, the optimization procedure works as follows:
1. The Gaussian process of the Bayesian optimization is initialized with an uninformative prior.
2. The following steps are iterated until the user finds a segmentation that is sufficiently accurate
   a. The current posterior likelihood over segmentation parameters (given by the current Gaussian process) is used together with an acquisition function to randomly sample a small set of new parameter settings. Here, any acquisition function can be used, it is preferred to employ an acquisition function that promotes a certain degree of diversity of the samples, e.g. batch expected improvements.
   b. Each sampled parameter set is used to create a segmentation of the physical particles in the input image 6.
   c. The current best segmentation is found by looking at all previously used parameter sets and taking the segmentation of the parameter set that achieves highest utility as modeled by the current Gaussian process.
   d. In a user interface each of the segmentations, i.e. those associated with the newly sampled parameter set as well as the current best segmentation, are shown to the user 1. The user 1 is asked to indicate among those displayed segmentations the one that she deems best.
   e. Once the user 1 has selected the best segmentation among those displayed, this induces a set of pairwise comparisons in the sense that the chosen segmentation is better then each of the other segmentations. With these pairwise comparisons the Gaussian process is updated.

### LIST OF REFERENCES

1 User
2 Computer/Control Unit
3 Memory
4 Display
5 Image Sensor/Microscope
6 Digital Microscopy Images
7 AI/Machine Learning Model
8 Software/Control Program
9 User Interface (GUI)
10 Active Pharmaceutical Ingredient particles (API)
11 System for Scanning Electron Microscopy (SEM)

1. A method of computer-based small particle measurement for drug formulation, comprising
creating digital microscopy images (6) of the small particles (10) used for the drug formulation by an image sensor (5) and providing the images to a computer (2) performing a measurement software (8),
wherein the software (8) segments the small particles (10) in the digital microscopy images (6) and calculates properties of the small particles (10) according to specific parameter sets,
wherein the software (8) samples different candidate parameter sets, applies them automatically for the segmentation and/or calculation process and shows the results via a display (4) to a user (1), and
wherein the user (1) picks the candidate parameter set with the best results, the software (8) establishes and trains an internal machine learning model (7) with this user feedback and applies the trained model (7) to reiterate the automatic segmentation and/or calculation and user feedback obtaining process until an optimal parameter set has been approved by the user (1).

2. The method according to claim 1, wherein Active Pharmaceutical Ingredient particles (API) (10) are used as small particles (10).

3. The method according to claim 1, wherein the measured properties comprise of the structure and fractality of the small API particle surface to determine the surface smoothness of small API particles and of the particle size distributions.

4. The method according to claim 1, wherein the small particles (10) comprise of highly variable particle shapes and sizes.

5. The method according to claim 1, wherein the digital microscopy images (6) are created under variable lighting conditions.

6. A system to perform a computer-based small particle measurement for drug formulation comprising:
a computer (2) performing a measurement software (8),
a display (4) to show information to a user (1),
means for the user (1) to input data and/or instructions to the software (8), and
an image sensor (5),
wherein the system (11) is arranged to create digital microscopy images (6) of the small particles (10) used for the drug formulation via the image sensor (5), to segmentate the small particles (10) in the digital microscopy images (6) and calculate properties of the small particles (10) via the software (8) according to specific parameter sets, and
wherein the software (8) samples different candidate parameter sets, applies them automatically for the segmentation and/or calculation process and shows the results via the display (4) to the user (1), and
wherein the user (1) picks the candidate parameter set with the best results, the software (8) establishes and trains an internal machine learning model (7) with this user feedback and applies the trained model (7) to reiterate the automatic segmentation and/or calculation and user feedback obtaining process until an optimal parameter set has been approved by the user (1).

7. The system according to claim 6, wherein the image sensor (5) is an scanning electron or bright field microscope creating microscopy images as digital images (6).

8. The system according to claim 6, wherein the software (8) comprise of either two connected software components, one component responsible for the particle segmentation and the other one for the property calculation, or of one software component performing both tasks.

9. The method according to claim 2, wherein the measured properties comprise of the structure and fractality of the small API particle surface to determine the surface smoothness of small API particles and of the particle size distributions.

10. The method according to claim 2, wherein the small particles (10) comprise of highly variable particle shapes and sizes.

11. The method according to claim 2, wherein the digital microscopy images (6) are created under variable lighting conditions.

12. The method according to claim 3, wherein the small particles (10) comprise of highly variable particle shapes and sizes.

13. The method according to claim 3, wherein the digital microscopy images (6) are created under variable lighting conditions.

14. The method according to claim 4, wherein the digital microscopy images (6) are created under variable lighting conditions.

15. The system according to claim 7, wherein the software (8) comprise of either two connected software components, one component responsible for the particle segmentation and the other one for the property calculation, or of one software component performing both tasks.
